(19) United States
(12) Patent Application Publication
(10) Pub. No.: US 2022/0226539 A1
(43) Pub. Date: Jul. 21, 2022

(54) COMPOSITE SCAFFOLD CONTAINING DFO AND RHBMP-2, PREPARATION METHOD AND USE THEREOF

(71) Applicant: EAST CHINA UNIVERSITY OF SCIENCE AND TECHNOLOGY, Shanghai (CN)

(72) Inventors: Yuan YUAN, Shanghai (CN); Haoyi NIU, Shanghai (CN); Hua HONG, Shanghai (CN); Yongsheng LI, Shanghai (CN); Lili SUN, Shanghai (CN)

(73) Assignee: EAST CHINA UNIVERSITY OF SCIENCE AND TECHNOLOGY, Shanghai (CN)

(21) Appl. No.: 17/199,835

(22) Filed: Mar. 12, 2021

(30) Foreign Application Priority Data
Jan. 18, 2021 (CN) 202110064199.4

(51) Int. Cl. A61L 27/44; A61L 27/56; A61L 27/54; A61K 38/18

(52) U.S. Cl. CPC A61L 27/44; A61L 27/56; A61L 27/54; A61L 2420/02; A61L 2430/02; A61L 2300/252; A61K 38/1875

(57) ABSTRACT

The present disclosure relates to a composite scaffold containing DFO and rhBMP-2 capable of synergistically stimulating bone formation, a preparation method and use thereof. The composite scaffold contains a matrix, a PEGS gel layer and rhBMP-2, wherein the matrix is an MBG scaffold grafted with DFO on the surface, the PEGS gel layer is carried on the surface of the matrix, and rhBMP-2 is carried inside the PEGS gel layer. In the present disclosure, the function of DFO and rhBMP-2 in vivo and in vitro can be regulated by precisely controlling the immobilization mode and spatial distribution of DFO and rhBMP-2 in the scaffold, and the all-round repair of “rapid enrichment of target cells—angiogenesis-guided bone” can be achieved.

The applicant claims the priority of Chinese application no. 202110064199.4 filed on Jan. 18, 2021.

### TECHNICAL FIELD

The present disclosure relates to the field of materials science and medicine, in particular to a composite scaffold, which contains MBG having micro-nano multilevel structure as matrix, is functionalized with PEGS and loaded with DFO and rhBMP-2, a preparation method and use thereof, especially for a scaffold material for bone disease patients with weak self-regeneration ability.

### BACKGROUND TECHNIQUE

The process of bone tissue repair and regeneration is mainly bone reconstruction and relates to a series of spatiotemporal coordination mechanisms during the reconstruction process, including the formation of a specific microenvironment, the recruitment of bone matrix cells, the reconstruction of blood vessels, and the induction of endoosteogenesis in situ. Therefore, the construction of a microenvironment that is helpful to stimulate target cell recruitment and vessel reconstruction is the key to quickly initiate the bone formation process, and it is the main means to optimize and strengthen guided bone regeneration materials. Studies have shown that the hypoxia microenvironment can activate hypoxia-inducible factor HIF-1α, thereby activating the expression of downstream VEGF, SDF-1 and other signal factors related to angiogenesis and stem cell recruitment. It can be seen that HIF-1α produced by the hypoxic microenvironment can stimulate multiple osteogenesis initiating functional factors, and provide new ideas for the rapid activation of target cell enrichment and blood vessel formation in the initial stage of bone repair. How to construct a hypoxic microenvironment is the key to the process. Recent studies have found that deferoxamine DFO used as anoxia mimetic agent in clinic can chelate iron ions to compete for the binding site between iron ions and α-ketoglutarate, reduce the activity of proline hydroxylase and inhibit degradation of HIF-1α and stabilize its activity, thereby effectively inducing angiogenesis/vascularization as well as endogenous bone regeneration and bone defect healing. However, high-dose of DFO not only has prominent problems such as high cytotoxicity and short intravascular half-life, but also has a certain negative impact on the osteogenic activity of rhBMP-2. Therefore, it is still necessary to study and design functional matrix materials that can load DFO and rhBMP-2 and precisely regulate the temporal and spatial distribution of DFO and rhBMP-2 in vitro and in vivo, thereby reducing negative effects.

### SUMMARY OF THE INVENTION

The object of the present disclosure is to provide an MBG composite scaffold with macropore/micropore/mesoporous hierarchical structure, spatiotemporally loading and sequentially releasing hypoxia analog drug DFO and growth factor rhBMP-2, a preparation method and use thereof.

The first aspect of the present disclosure provides a composite scaffold carrying DFO and rhBMP-2, wherein the composite scaffold contains a matrix, a PEGS gel layer and rhBMP-2, wherein the matrix is an MBG scaffold grafted with DFO on the surface; the PEGS gel layer is carried on the surface of the matrix; and rhBMP-2 is contained inside the PEGS gel layer.

In another preferred embodiment, the surface of the substrate has two PEGS gel layers, wherein the first PEGS gel layer is on the surface of the substrate, and the second PEGS gel layer is on the surface of the first PEGS gel layer, and rhBMP-2 is in the second PEGS gel layer.

The MBG composite scaffold with macropore/micropore/mesoporous hierarchical structure, spatiotemporally loading and sequentially releasing hypoxia analog drug DFO and growth factor rhBMP-2 of the present disclosure uses hierarchical mesoporous bioglass as matrix, wherein DFO and rhBMP-2 are immobilized on the inner and outer surfaces of the mesopores through glutaraldehyde chemical cross-linking and azidated PEG polyglycerol sebacate (PEGS).

The composite scaffold of the present disclosure is a scaffold capable of simulating the hypoxia microenvironment and efficiently and stably expressing the hypoxia-inducible factor HIF-1α. By chemically grafting the drug DFO, it can enhance the expression level of various signal factors related to angiogenesis and progenitor cell recruitment in situ.

In another preferred embodiment, rhBMP-2 and DFO are loaded in the MBG scaffold, respectively and separated by a PEGS coating, and rhBMP-2 and DFO are simultaneously present on the pore surface of the hierarchical pores.

In another preferred embodiment, the MBG scaffold is a hierarchical pore MBG scaffold with 200 μm-500 μm macropores, 1-3 μm micropores and 2-5 nm mesopores.

In another preferred embodiment, the hierarchical pore MBG scaffold has a good interconnected pore structure.

In another preferred embodiment, the surface grafting refers to grafting on the inner and outer surfaces, that is, both the inner and outer surfaces of the MBG scaffold are grafted with DFO.

In another preferred embodiment, the surface of the substrate refers to the inner and outer surfaces of the substrate, that is, both the inner and outer surfaces of the substrate are loaded with a PEGS coating.

In another preferred embodiment, the iron ion chelating capacity of the composite scaffold is 5-20 μmol/g.

In another preferred embodiment, the iron ion chelating capacity of the composite scaffold is 8-15 μmol/g, preferably 10 μmol/g.

In another preferred embodiment, the release of rhBMP-2 factor in vitro within 30 days is 10-24 wt %.

In another preferred embodiment, the thickness of the PEGS gel layer is 1-2 μm.

In another preferred embodiment, the loading amount of the rhBMP-2 is 0.005-0.1 μg rhBMP-2 per mg of scaffold.

In another preferred embodiment, the loading amount of the rhBMP-2 is 0.01-0.08 μg rhBMP-2 per mg of scaffold, preferably 0.02-0.05 μg rhBMP-2 per mg of scaffold.

The second aspect of the present disclosure provides a preparation method of the composite scaffold according to the first aspect, wherein the preparation method comprises the following steps:

i) providing a MBG scaffold and PEGS prepolymer including azidated PEGS prepolymer and alkynylated PEGS prepolymer;

ii) grafting DFO on the surface of the MBG scaffold to obtain a matrix;

iii) mixing the azidated PEGS prepolymer with rhBMP-2 to obtain a mixture; coating the mixture on the surface of the matrix obtained in step ii); and then coating the alkynylated PEGS prepolymer to form PEGS gel layer with rhBMP-2 loaded inside, thereby obtaining the composite scaffold;

or coating the azidated PEGS prepolymer solution on the MBG-DFO scaffold and then coating the alkynylated PEGS prepolymer solution to form a PEGS gel isolation layer, and then loading rhBMP-2 to form PEGS gel layer with rhBMP-2 loaded inside, thereby obtaining the composite scaffold.

In the present disclosure, the MBG-DFO scaffold is obtained by chemically grafting the hypoxia analog drug on the inner and outer surfaces of the mesopores of the MBG scaffold by siloxane coupling agent-glutaraldehyde-DFO. In another preferred embodiment, when DFO is grafted onto the surface of the MBG scaffold in step ii), DFO is covalently connected by the schiff base reaction of APTMS, glutaraldehyde and DFO.

In another preferred embodiment, in step ii), DFO is grafted onto the surface of the MBG scaffold by the following steps:

ii-1) reacting MBG scaffold with 3-aminopropyltrimethoxysilane (APTMS) to obtain an MBG scaffold with aminated surface, MBG-NH₂;

ii-2) reacting MBG-NH₂ with glutaraldehyde to obtain an intermediate product, MBG-CHO scaffold;

ii-3) reacting MBG-CHO scaffold with DFO, and grafting DFO on the surface of the MBG scaffold to obtain the MBG-DFO scaffold.

In another preferred embodiment, in step ii-1), the amount of added 3-aminopropyltrimethoxysilane (APTMS) is 0.5-2 ml/g MBG scaffold.

In another preferred embodiment, the MBG scaffold is dried and then immersed in anhydrous toluene, then 3-aminopropyltrimethoxysilane (APTMS) is added and dissolved in toluene, and the mixture is reacted at 80° C. for 24 hours. The supernatant is discarded, the scaffold is washed separately with toluene and absolute ethanol for 3 times and then the scaffold is placed in a vacuum oven at 60° C. for 24 hours to obtain an MBG scaffold with aminated surface (MBG-NH₂).

In another preferred embodiment, in step ii-2), 1-4 ml of 25% glutaraldehyde is added to each gram of MBG-NH₂.

In another preferred embodiment, the MBG scaffold with aminated surface (MBG-NH₂) is immersed in ultrapure water, glutaraldehyde is added, and the mixture is stirred and reacted at 37° C. for 6 hours to obtain the intermediate product MBG-CHO scaffold. The scaffold is washed 3 times with ultrapure water and dried.

In another preferred embodiment, in step ii-3), the amount of added DFO is 0.1-0.8 g DFO/g MBG-CHO scaffold, preferably 0.5 g DFO/g MBG-CHO scaffold.

In another preferred embodiment, the MBG-CHO scaffold and DFO are added to ultrapure water and reacted at 37° C. for 6 hours, then the scaffold is washed with ultrapure water and placed in a vacuum oven at 60° C. to obtain the MBG-DFO scaffold.

In another preferred embodiment, the azidated PEGS prepolymer is obtained by the following steps:

(a-1) reacting PEG with sebacoyl dichloride and triethylamine to obtain sebacoyl dichlorinated PEG;

reacting sebacoyl dichlorinated PEG with glycidol and triethylamine to obtain a long-chain monomer with a ring at both ends;

reacting the monomer with sebacic acid and tetrabutylammonium bromide through ring-opening reaction to obtain PEGS molecules with exposed hydroxyl in the side chain, labeled as HPEGS;

reacting HPEGS with maleic anhydride to obtain maleic acid-functionalized PEGS, labeled as HPEGS-M;

(a-2) adding 3-azidopropylamine and triethylamine to the separation product of dicyclohexylcarbodiimide, N-hydroxysuccinimide and HPEGS-M to obtain the azidated PEGS prepolymer, labeled as HPEGS-Az.

In another preferred embodiment, the functionalized PEGS prepolymer, i.e., azidated PEGS prepolymer (PEGS-Az) and alkynylated PEGS prepolymer (PEGS-DBCO) are dissolved in a PBS solution, respectively. The azidated PEGS prepolymer solution is coated onto the MBG-DFO scaffold by dropping and then the alkynylated PEGS prepolymer is coated, a PEGS gel isolation layer is formed within minutes.

In another preferred embodiment, the azidated PEGS prepolymer solution and the alkynylated PEGS prepolymer solution are successively coated on the MBG-DFO scaffold to form a PEGS gel isolation layer, and then the rhBMP-2 is loaded by the following steps: the azidated PEGS prepolymer is uniformly mixed with rhBMP-2 and the mixture is coated on the PEGS gel isolation layer, and then the alkynylated PEGS prepolymer solution is coated to form a PEGS gel layer with rhBMP-2 loaded inside.

In another preferred embodiment, the concentration of the prepolymer solution is 20-40 wt %, preferably 30 wt %.

In another preferred embodiment, the alkynylated PEGS prepolymer is obtained by the following steps:

(a-1) reacting PEG with sebacoyl dichloride and triethylamine to obtain sebacoyl dichlorinated PEG;

reacting sebacoyl dichlorinated PEG with glycidol and triethylamine to obtain a long-chain monomer with a ring at both ends;

reacting the monomer with sebacic acid and tetrabutylammonium bromide through ring-opening reaction to obtain PEGS molecules with exposed hydroxyl in the side chain, labeled as HPEGS;

reacting HPEGS with maleic anhydride to obtain maleic acid-functionalized PEGS, labeled as HPEGS-M;

(a-2) reacting HPEGS-M with dicyclohexylcarbodiimide and N-hydroxysuccinimide, and then adding aminated diphenylcyclooctyne and triethylamine to react to obtain the alkynylated PEGS prepolymer, labeled as HPEGS-DBCO.

The third aspect of the present disclosure provides a method for repairing bone tissue comprising the step of administering of the composite scaffold according to the first aspect to a subject in need thereof. The bone tissue repair means bone defect filling repair or guided bone regeneration.

The fourth aspect of the present disclosure provides a composition carrier containing the composite scaffold according to the first aspect and a growth factor, or drug.

Based on the combination of rhBMP-2 and the hypoxia mimic deferoxamine (DFO) capable of activating the expression function of hypoxia inducible factor HIF-1α and various downstream factors, a material for in situ stimulation of regeneration is constructed in the present disclosure. The composite scaffold is based on mesoporous bioglass with a hierarchical structure, and DFO and rhBMP-2 are immobilized on the inner and outer surfaces of the mesopores through glutaraldehyde chemical crosslinking and azidated PEG polyglycerol sebacate (PEGS), respectively. By precisely controlling the immobilization mode and spatial distribution of DFO and rhBMP-2 in the material, their functions in vivo and in vitro are regulated. All-round repair of “rapid enrichment of target cells—angiogenesis-guided bone in situ” is achieved by activating the expression function of hypoxia inducible factor HIF-1α and its downstream factors as well as synergizing with rhBMP-2. From the cellular level and experimental results of in situ defect repair in animals, the regulation of the spatiotemporal distribution and sequential release of DFO and rhBMP-2 on cell recruitment, angiogenesis, and osteogenic differentiation is fully clarified and a theoretical model to stimulate bone regeneration by simulating the hypoxic microenvironment and synergizing with rhBMP-2 is proposed. Such hypoxia analog drug/rhBMP-2 composite scaffold promotes the bone repair process and improves the quality of bone repair, and is a bone repair scaffold with clinical application prospects.

It should be understood that within the scope of the present disclosure, the above-mentioned technical features of the present disclosure and the technical features specifically described in the following (such as the examples) can be combined with each other to form a new or preferred technical solution. Each feature disclosed in the specification can be replaced by any alternative feature that provides the same, equal or similar purpose. Due to space limitations, they will not be repeated one by one.

### BRIEF DESCRIPTION OF THE DRAWINGS

By reviewing the following detailed description of the non-limiting embodiments with reference to the accompanying drawings, other features, purposes and advantages of the present disclosure will become more apparent.

FIG. 1 is a schematic diagram of the structure of the hierarchical mesoporous bioglass composite scaffold material loaded with hypoxia analog drug DFO and the growth factor rhBMP-2 in the form of isolation of the present disclosure.

FIG. 2 is a characterization diagram of the scaffold: (A) the infrared spectrum of MBG, MBG grafted with APTMS (MBG-NH₂), further grafted with glutaraldehyde (MBG-CHO) and further grafted DFO (MBG-DFO), from top to bottom; (B) solid-state NMR spectra of MBG (bottom) and MBG-DFO (top); (C) IR spectra of PEGS prepolymer and hydrogel, which are PEGS-Az prepolymer, PEGS-DBCO prepolymer, and PEGS hydrogel from top to bottom; (D) PEGS hydrogel morphology; (E) sustained release curve of rhBMP-2 from B@M and D@M+B@P composite scaffolds; (F) iron ion chelating ability of the D@M+B@P composite scaffold detected by EDS surface scanning; (G) the macroporous/mesoporous structure and morphology of the composite scaffold analyzed by SEM and TEM.

FIG. 3 shows the ability of each group of scaffold materials to simulate the hypoxic microenvironment analyzed by HIF-1α protein immunofluorescence staining.

FIG. 4 shows the in vitro cell recruitment ability of each group of scaffold materials analyzed by BMSCs and HUVECs migration experiments.

FIG. 5 shows the ability of each group of scaffold materials to promote angiopoiesis and osteogenesis of cells: (A) bud growth of HUVECs in vitro and expression of angiogenesis-related protein VEGF; (B) ALP staining.

FIG. 6 shows the ability of the B@M and D@M+B@P composite scaffolds to promote in vivo angiogenesis and osteogenesis analyzed by repair experiment of distal femoral defect in rats: (A) Micro-CT for angiogenesis; (B) Micro-CT for bone defect repair.

FIG. 7 is an immunofluorescence staining image of osteogenic-related protein ColI on tissue sections at 2, 4, and 8 weeks after surgery to analyze the bone repair ability of each group of composite scaffolds.

### DETAILED DESCRIPTION OF THE INVENTION

The inventors of the present disclosure have conducted extensive and intensive research and found that the combination of DFO and rhBMP-2 can achieve rapid stimulation and efficient osteogenesis, wherein the hypoxic microenvironment created by DFO can activate multiple growth factors simultaneously. Taking into account the toxicity of free DFO to the body and its potential impact on the osteogenic activity of rhBMP-2, DFO is chemically grafted onto the surface of the MBG scaffold via Schiff base reaction, and separated from rhBMP-2 by PEGS hydrogel with excellent biocompatibility, and furthermore, the sustained release of rhBMP-2 is achieved by encapsulating rhBMP-2 with hydrogel, thereby achieving the different spatiotemporal distribution in the material and sequential release in vivo of DFO and rhBMP-2. The inventors explore the law and mechanism of DFO and rhBMP-2 in synergistic regulation on tissue formation, and prepare a composite scaffold that can quickly enrich osteoblast-related cells in the process of bone tissue repair and promote their differentiation and blood vessel formation to achieve “rapid excitation and high efficient osteogenesis”. On this basis, the present disclosure has been completed.

Terms

Unless otherwise defined, all technical and scientific terms used herein have the same meaning as those commonly understood by those of ordinary skill in the art to which the present disclosure belongs.

As used herein, when used in reference to a specifically recited value, the term “about” means that the value can vary from the recited value by no more than 1%. For example, as used herein, the expression “about 100” includes all values between 99 and 101 (e.g., 99.1, 99.2, 99.3, 99.4, etc.).

As used herein, the term “contain” or “include (including)” can be open, semi-closed, and closed. In other words, the term also includes “substantially consisting of” or “consisting of”.

As used herein, the terms “MBG” and “hierarchical mesoporous bioglass” can be used interchangeably.

As used herein, the terms “F127” and “polyoxyethylene polyoxypropylene ether” can be used interchangeably.

As used herein, the terms “PEGS” and “PEGylated polyglyceryl sebacate” can be used interchangeably.

As used herein, the terms “DFO” and “deferoxamine” can be used interchangeably.

As used herein, the terms “PEG” and “polyethylene glycol” can be used interchangeably.

As used herein, in the terms “B@M” and “D@M+B@P”, @ represents load and +represents encapsulation.

In the present disclosure, the MBG scaffold is loaded with rhBMP-2 to obtain a B@M scaffold; and the MBG-DFO scaffold is coated with a PEGS hydrogel encapsulating rhBMP-2 to obtain a D@M+B@P scaffold.

As used herein, the terms “phosphate buffer” and “PBS” can be used interchangeably.

As used herein, the terms “rat mesenchymal stem cells” and “rBMSCs” can be used interchangeably.

Composite Scaffold

At present, the role of hypoxia mimic drug DFO in vascularization and bone precursor cell recruitment has been widely recognized, and rhBMP-2 is currently recognized as a growth factor that induces osteogenic differentiation. It will be an ideal solution for constructing stimulating renewable materials to design and construct new functional matrix materials based on different functions and characteristics of these two drugs and factors (i.e., DFO and rhBMP-2 approved by FDA). However, high-dose DFO not only has prominent problems such as greater cytotoxicity and shorter intravascular half-life, but also has a certain negative impact on the osteogenic activity of rhBMP-2. Therefore, the present disclosure develops a composite scaffold that can be loaded with DFO and rhBMP-2 and precisely regulate the spatiotemporal distribution of DFO and rhBMP-2 in vitro and in vivo, with the aim of reducing negative effects and maximizing the synergistic guidance of bone tissue regeneration.

The composite scaffold of the present disclosure has the ability of activating the HIF signal pathway and synergistic osteogenesis. The effects of MBG scaffolds, MBG scaffolds only loading rhBMP-2, and MBG scaffolds spatiotemporally loading and sequentially releasing DFO and rhBMP-2 on scaffold on the recruitment and migration of mesenchymal stem cells and vascular endothelial cells, angiogenic differentiation in vitro and osteogenic differentiation as well as bone tissue repair in vivo are studied in the present disclosure, based on the difference in the loading of drugs and factors. Finally, a new type of high-efficiency bone repair biomaterials is obtained, the basic law and mechanism of synergistic promotion of osteogenesis are studied in depth, and a theoretical model of in situ bone regeneration induced synergistically by hypoxic microenvironment, rhBMP-2 and scaffold materials is proposed and established. The invention closely combines clinical needs and has clinical application prospects.

Preparation Method

In the present disclosure, the preparation method of pegylated polyglyceryl sebacate prepolymer includes the following steps.

PEG with a molecular weight of 600-3000 g/mol, sebacoyl dichloride, triethylamine, and glycidol are weighed in an anhydrous and oxygen-free glove box. PEG is reacted with a certain amount of sebacoyl dichloride and triethylamine in a low temperature environment (optimally 0 degrees) for 24 hours to obtain sebacoyl dichlorinated PEG.

Then the product and glycidol are uniformly mixed in a toluene solution, and reacted with triethylamine in a low temperature environment to obtain a long-chain monomer with a ring at both ends.

The monomer is further dissolved with sebacic acid and tetrabutylammonium bromide in the DMF solution, and a PEGS polymer with abundant exposed hydroxyl in the side chain (HPEGS) is obtained through a ring-opening reaction.

The purified PEGS high molecular polymer is obtained by dialysis purification. The PEGylated polyglyceryl sebacate prepolymer is used for subsequent azidation and alkynylation modification.

The preparation method of maleic acid functionalized PGES (HPEGS-M) includes the following steps.

PEG with a molecular weight of 600-3000 g/mol, sebacoyl dichloride, triethylamine, and glycidol are weighed in an anhydrous and oxygen-free glove box. PEG is reacted with a certain amount of sebacoyl dichloride and triethylamine in a low temperature environment (optimally 0 degrees) for 24 hours to obtain sebacoyl dichlorinated PEG.

Then the product and glycidol are uniformly mixed in a toluene solution, and reacted with triethylamine in a low temperature environment to obtain a long-chain monomer with a ring at both ends.

The monomer is further dissolved with sebacic acid and tetrabutylammonium bromide in the DMF solution, and a PEGS polymer with abundant exposed hydroxyl in the side chain (HPEGS) is obtained through a ring-opening reaction. The HPEGS molecule and the same amount of maleic anhydride are mixed in the dimethylformamide (DMF) solution to react to produce maleic acid functionalized PEGS (HPEGS-M).

The preparation method of azide group functionalized HPEGS-Az includes the following steps.

Dicyclohexylcarbodiimide (DCC) and N-hydroxysuccinimide (NHS) are dissolved in dimethyl sulfoxide (DMSO), add the mixture is added to the DMSO solution containing HPEGS-M under an argon atmosphere and is set at room temperature to obtain precipitates. 3-azidopropylamine and triethylamine are added to the separation product to obtain HPEGS-Az.

The preparation method of the alkynylated PEGS prepolymer HPEGS-DBCO includes the following steps.

HPEGS-M is added to the DCC/NHS-containing DMSO solution under an argon atmosphere, and aminated diphenylcyclooctyne and triethylamine are further added to react at room temperature to obtain HPEGS-DBCO.

In another preferred embodiment, the molecular weight of the PEG is 600-3000 g/mol, and the carboxyl grafting rate is 20%-60%. The weight-average molecular weight of PEGS prepolymer HPEGS-M measured by gel permeation chromatography is about 30,000 Da.

The present disclosure will be further explained below in conjunction with specific examples. It should be understood that these examples are only used to illustrate the present disclosure and not to limit the scope of the present disclosure. The experimental methods without specific conditions in the following examples are usually in accordance with conventional conditions or in accordance with the conditions suggested by the manufacturer. Unless otherwise specified, percentages and parts are percentages by weight and parts by weight.

Unless otherwise defined, all professional and scientific terms used herein have the same meaning as those familiar to those skilled in the art. In addition, any methods and materials similar or equivalent to the contents described herein can be applied to the method of the present disclosure. The preferred embodiments and materials described herein are for demonstration purposes only.

### Example 1

Preparation of Alkynylated PEGS Prepolymer and Alkynylated PEGS Prepolymer

5 g of epoxidized PEG (Mn=500 D), 2.02 g of sebacic acid and 44 mg of tetrabutylammonium bromide as a catalyst were weight in an anhydrous and oxygen-free glove box, dissolved and mixed in dimethylformamide (DMF), and heated (optimally at 100 degrees) and reacted for 72 hours to obtain a PEGS macromolecule with abundant exposed hydroxyl groups in the side chain. The number average molecular weight of PEGS polymer measured by gel permeation chromatography is about 10000 Da. The PEGS (7 g) and maleic anhydride (2 g) having an equimolar amount of hydroxyl were mixed in the DMF solution and reacted at 100° C. for 1 hour to generate maleic acid functionalized PEGS.

The preparation method of azidated PEGS includes the following steps.

Under an argon atmosphere, 4.12 g of dicyclohexylcarbodiimide (DCC) and 2.3 g of N-hydroxysuccinimide (NHS) were dissolved in dimethyl sulfoxide (DMSO), added to DMSO solution containing 9 g of the above maleic acid-functionalized PEGS and mixed thoroughly. 100 mg of 3-azidopropylamine and 100 mg of triethylamine were added to react overnight at room temperature to obtain azidated PEGS.

The preparation method of alkynylated PEGS is similar to that of azidated PEGS, including the following steps.

Under an argon atmosphere, 4.12 g of dicyclohexylcarbodiimide (DCC) and 2.3 g of N-hydroxysuccinimide (NHS) were dissolved in dimethyl sulfoxide (DMSO), added to DMSO solution containing 9 g of the above maleic acid-functionalized PEGS and mixed thoroughly. 100 mg of aminated dibenzocyclooctyne and 100 mg of triethylamine were added to react overnight at room temperature to obtain alkynylated PEGS.

### Example 2

This example relates to the controllable preparation of MBG, B@M and D@M+B@P scaffolds

(a) Preparation of MBG scaffold with macroporous/microporous/mesoporous hierarchical structure

MBG scaffold with macroporous/microporous/mesoporous hierarchical structure was prepared by referring to Niu H, Lin D, Tang W, et al. Surface topography regulates osteogenic differentiation of MSCs via crosstalk between FAK/MAPK and ILK/β-catenin pathways in a hierarchically porous environment. ACS Biomaterials Science & Engineering, 2017. PU sponge, polyacrylic acid microspheres, F127 as macroporous, microporous, and mesoporous templates were used to prepare hierarchical pore MBG scaffolds with a mesopore size of 2-20 nm.

(b) Preparation of MBG-DFO scaffold

Amination of MBG: 200 mg of MBG scaffold was dried and immersed in 100 mL of anhydrous toluene, then 600 μL of 3-aminopropyltrimethoxysilane (APTMS) was added and dissolved in toluene. The mixture was reacted at 80° C. for 24 hour. The supernatant was discarded, the scaffold was washed separately with toluene and absolute ethanol for 3 times, and then the scaffold was placed in a vacuum oven at 60° C. for 24 hours to obtain an MBG scaffold with aminated surface, MBG-NH₂.

Synthesis of MBG-DFO: 200 mg of MBG-NH₂ scaffold obtained from the above reaction was immersed in 100 mL ultrapure water, 4 mL glutaraldehyde (25%) was added, and the mixture was stirred and reacted at 37° C. for 6 hours to obtain the intermediate product MBG-CHO scaffold, which was then washed 3 times with ultrapure water and dried. Then, 200 mg of MBG-CHO scaffold and 100 mg of DFO were added to 100 mL of ultrapure water, and reacted at 37° C. for 6 hours. Then, the scaffold was washed with ultrapure water and dried in a vacuum oven at 60° C. to obtain MBG-DFO scaffold.

(c) Preparation of B@M scaffold (rhBMP-2-loading MBG)

The saturated adsorption volume of the scaffold (10 mm×10 mm×3 mm³) measured with PBS was 250 μL. 1 μg of rhBMP-2 was dissolved in 125 μL of 30% alkynylated PEGS prepolymer and coated on the MBG scaffold. Then 125 μl of 30% azidated PEGS prepolymer was coated and cross-linked with alkynylated PEGS prepolymer for a few minutes to obtain the MBG scaffold loaded with rhBMP-2, named B@M.

For the preparation of D@M+B@P scaffold, a layer of PEGS hydrogel coating was firstly coated on the surface of MBG-DFO scaffold, and then the rhBMP-2-containing hydrogel was coated on the surface. Specifically, 125 μl of 30% alkynylated PEGS prepolymer was coated on the MBG-DFO scaffold. Then 125 μl of 30% azidated PEGS prepolymer was coated and cross-linked with alkynylated PEGS prepolymer for a few minutes to obtain a PEGS hydrogel coating. 1 μg of rhBMP-2 was dissolved in 125 μL of 30% alkynylated PEGS prepolymer and the mixture was coated on the MBG-DFO scaffold coated with PEGS hydrogel. And then 125 μl of 30% azidated PEGS prepolymer was coated and cross-linked with alkynylated PEGS prepolymer to obtain a D@M+B@P scaffold.

As shown in FIG. 1, a composite scaffold loaded with DFO and rhBMP-2 is obtained. The thickness of the coated gel can be observed and calculated from the SEM image, and is about 2 μm.

### Example 3

This example relates to the characterization of the DFO grafting and iron ion chelating ability of the composite scaffold and the characterization of the sustained release ability of rhBMP-2 of the composite scaffold.

In order to test whether the azide group functionalized HPEGS-Az and alkynylated PEGS prepolymers HPEGS-DBCO prepared in Example 1 and the MBG-DFO prepared in Example 2 were successfully synthesized, the chemical composition of the material was tested by an infrared spectrometer (Nicolet 6700, USA) and by the method of attenuated total reflection. The product was analyzed by the hot melt coating method, and the collection range was 4000-800 cm′. The chemical structure of the material was analyzed by analyzing the characteristic vibration peaks of each group in the infrared spectrum. In addition, the chemical structure of MBG-DFO was analyzed by carbon nuclear magnetic resonance method. For the characterization of iron ion chelating ability, the MBG-DFO scaffold was quantitatively and qualitatively analyzed by inductively coupled plasma-optical emission spectrometer (ICP-OES) and energy spectrum analysis (EDS).

For the characterization of the sustained-release ability of rhBMP-2, the B@M scaffold (20 mg) containing 2 μg of rhBMP-2 was freeze-dried in vacuum at −40° C. overnight, and soaked in PBS. The content of rhBMP-2 in the solution was tested on day 1, 2, 3, 4, 7, 10, 14, 21, 28, respectively. The content of rhBMP-2 was determined by enzyme-linked immunosorbent assay ELISA to make a release curve.

$$Cumulative release percentage = \frac{Released rhBMP - 2}{Loaded rhBMP - 2} \times 100 \%$$

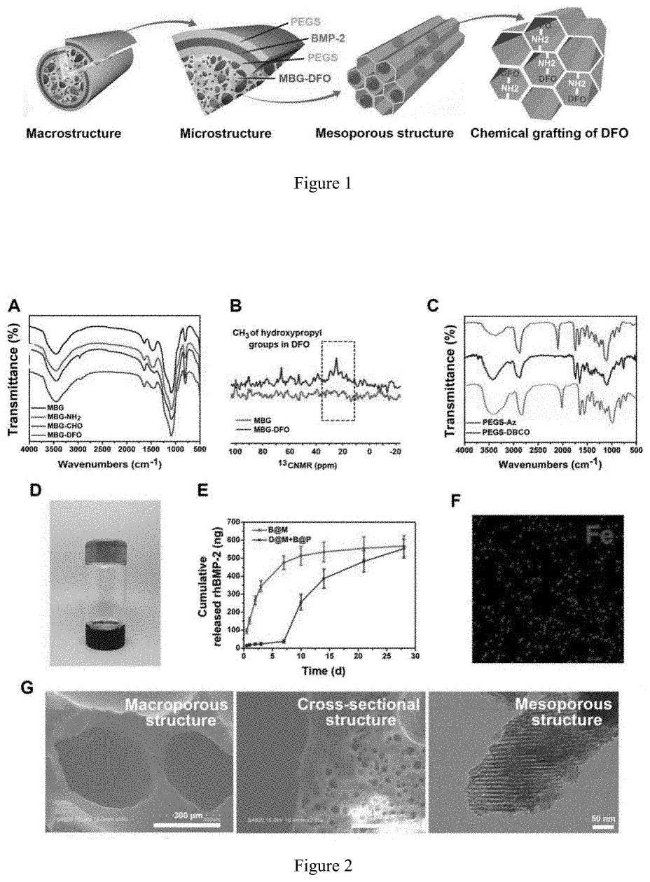

It can be seen from FIG. 2A-C, DFO has been successfully grafted onto the MBG scaffold, and functionalized PEGS (azide group functionalized HPEGS-Az and alkynylated PEGS prepolymer HPEGS-DBCO) have also been successfully synthesized.

As shown in FIG. 2E, both the B@M scaffold and D@M+B@P scaffold can release rhBMP-2 slowly, and the D@M+B@P scaffold has a better sustained release effect. The cumulative release rate of rhBMP-2 in 28 days is about 20%.

30% of alkynylated PEGS and azidated PEGS was prepared respectively, and mixed for several minutes to prepare gel. It can be seen from FIG. 2D that the functionalized PEGS prepolymer can form a hydrogel.

FIG. 2F is an EDS graph of iron ion chelation. After grafting DFO, the scaffold has a strong iron ion chelating ability. According to the adsorption quantitative test, the chelating ability is about 10 μmol/g.

FIG. 2G is the SEM image of the D@M+B@P composite scaffold, showing that the scaffold still has a pore structure after being composited.

### Example 4

This example relates to the ability of each group of scaffold materials to simulate the hypoxic microenvironment

In order to study the ability of each group of scaffold materials to simulate the hypoxic microenvironment, the expression of proteins related to the hypoxic environment of rBMSCs was detected by fluorescent staining of HIF-1α. The rBMSCs were seeded on the scaffolds in each group at a density of 1×10⁵ cells/well, and the cells were fixed after 24 hours of culture and observed by immunofluorescence staining.

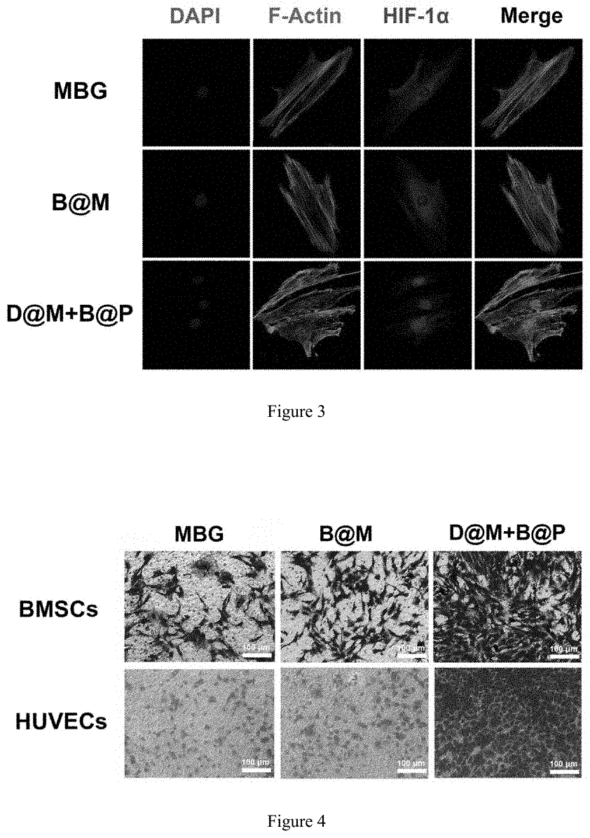

As shown in FIG. 3, the distribution of HIF-1α protein and aggregation in nucleus in rBMSC cells were studied. The results showed that there was little difference between MBG and B@M groups. HIF-1α protein was distributed in a large amount in the cytoplasm, and almost no expression was present in the nucleus. However, almost all HIF-1α proteins aggregated in the nucleus in the D@M+B@P group, indicating that the HIF-1α had the best aggregation in nucleus and expression ability in D@M+B@P group.

### Example 5

This example relates to the in vitro cell recruitment ability of each group of scaffold materials

In order to explore the in vitro cell recruitment ability of each group of scaffold materials, BMSCs and HUVECs were used as the research objects. The cell recruitment abilities for BMSCs and HUVECs of each group of materials were determined by using a polycarbonate membrane Transwell 24-well plate (membrane pore size: 8 μm, Corning, USA). The cells were digested and resuspended, and 2% FBS was added to the cell suspension. The cells were seeded into the upper chamber of Transwellat a cell density of 1×10⁴ cells/well (100 μL per well). Each group of scaffold materials and 2% FBS medium were added to the lower chamber. After the cells were cultured for 24 hours, the medium was removed from the upper chamber, and the cells were carefully wiped off with a cotton swab from the upper chamber. The cells in the lower chamber were photographed by using a microscope and the number of cells in each field was counted.

As shown in FIG. 4, compared with the MBG group and the B@M group, in the D@M+B@P group, the migration number of rBMSCs and HUVECs was significantly increased, and early stem cell recruitment and endothelial recruitment was promoted. The results show that grafted DFO can induce stem cells and endothelial cells migration, and DFO and rhBMP-2 have a synergistic effect. The early recruitment of stem cells can be significantly promoted by accurately regulating the spatiotemporal distribution of DFO and rhBMP-2, thereby providing a good cellular microenvironment for subsequent osteogenic differentiation.

### Example 6

This example relates to the ability of each group of scaffold materials to promote angiogenesis and osteogenesis in vitro

The ability of each group of scaffold materials to promote angiogenesis in vitro was in vitro analyzed through the ability of human umbilical vein endothelial cells to form blood vessel. HUVECs were seeded at a density of 2×10⁴ cells/well on each group of scaffolds in a 24-well plate, the medium was replaced every 2 days, and the cells were cultured for 7 days. Then the media was aspirated, the scaffold was washed 3 times with PBS, 200 μL of trypsin was added to the scaffold, and the cell digestion solution was collected. The matrigel without growth factors (BD Biosciences) was thawed at 4° C., and 100 μL of Matrigel was added to a 48-well plate on ice. Then the 48-well plate was placed at 37° C. for 30 minutes to form gel. The above cell digestion suspension was seeded at a cell density of 2×10⁴ cells/well on Matrigel.

The well plate was placed in a 37° C. cell incubator for 4 hours, the bud formation was observed through an optical microscope, and the total capillary length and the number of bifurcation points in each area were calculated by using NIH Image J 1.45 software. The expression of angiogenesis-related proteins in HUVECs was detected by fluorescent staining of VEGF protein. HUVECs were seeded on the scaffolds in each group at a density of 1×10⁵ cells/well. After the cells were cultured for 24 hours, the scaffold was washed 3 times with PBS, 200 μL of trypsin was added to the scaffold, and the cell digestion solution was collected and resuspended in a new 24-well plate, added with the medium and cultured for 24 hours. Then the media was aspirated, the scaffold was washed with PBS, and 1 mL of 2.5% glutaraldehyde was added to each well to fix the cells for 15 minutes. The scaffold was washed 3 times with PBS and then 1 mL of 0.1% Triton X-100 solution was added to each well to permeabilize the cells for 15 minutes. Then 1 mL of 5% BSA solution was added to each well, and the cells were incubated for 1 hour. After the blocking solution was sucked off, a stock solution of VEGF primary antibody was diluted at 1:500 with BSA solution and added to the well plate, and the cells were stained overnight at 4° C.

Then, the primary antibody was sucked off and the well was washed 3 times, and the secondary antibody solution labeled with Alexa Fluor®647 (diluting the stock solution of secondary antibody with BSA solution at 1:500) was added and incubated for 2 hours at room temperature in the darkness. The cytoskeleton of HUVECs was fluorescently stained with FITC-Phalloidin (Sigma, St Louis, USA). In a dark environment, 1 mL of FITC-Phalloidin solution at a concentration of 5 μg/mL was added to each well and incubated at room temperature for 45 minutes, and then the well was washed 3 times with PBS. The staining of cells in each group was observed by a confocal laser microscope (CLSM, A1, Nikon, Japan).

For the osteogenic activity of each group of scaffolds, the osteoinductive effects of MBG, B@M and D@M+B@P scaffolds on rBMSC were studied by detecting the activity of alkaline phosphatase ALP. The sterilized scaffold was placed in a 24-well plate, and 1 mL of α-MEM was added to each well to infiltrate the scaffold for 24 hours. Then the medium was sucked off, and the rBMSCs suspension was seeded on each scaffold in a 24-well plate at a density of 2×10⁴ cells/well, and cultured in a 37° C. incubator. The medium was replaced with fresh medium every 2 days. After the cells were cultured for 3 days and 7 days, the ALP activity was detected.

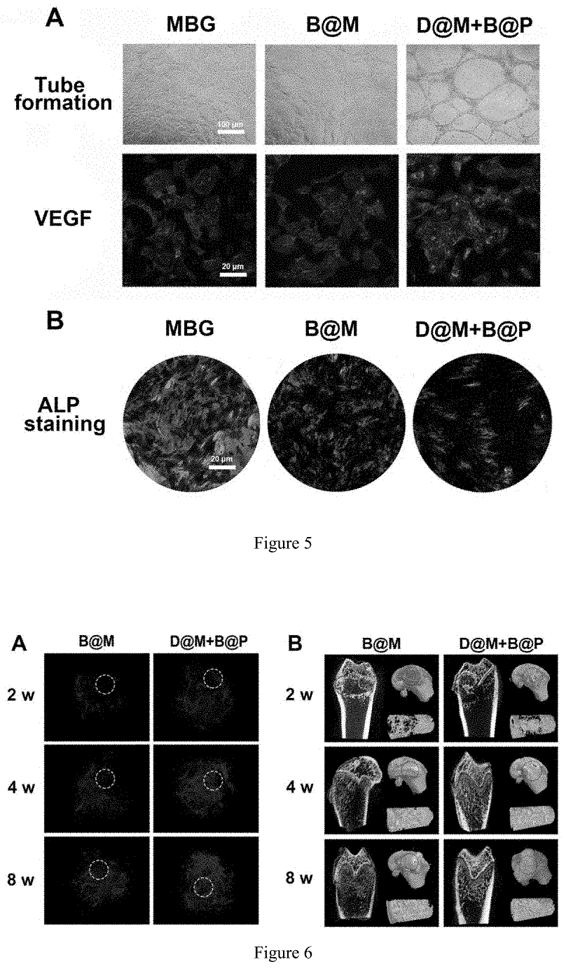

As shown in FIG. 5A, the vascularization ability was detected by tube-forming images of endothelial cells and immunofluorescence staining of VEGF protein. After 4 hours of culture in the MBG group, HUVECs did not show obvious bud growth. In the B@M group, the communication between the cells was enhanced, and the embryonic buds had already appeared. However, the occurrence of buds in the D@M+B@P group was very significant, the filopodia between the cells were connected and formed into a tube. The D@M+B@P group had the best bud growth ability. The expression of VEGF in HUVECs cultured on MBG scaffolds was weak; compared with MBG group, the expression of VEGF in B@M group cells increased slightly, but there was no significant difference, while D@M+B@P group further promoted the expression of VEGF and had the highest VEGF expression.

As shown in FIG. 5B, the expressions of ALP in the experimental group loaded with rhBMP-2 (B@M and D@M+B@P) were significantly higher than that of MBG.

### Example 7

This example relates to the characterization of in situ repair of rat femoral defect with composite scaffold

The bone repair ability of different scaffolds in vivo was studied by using a rat distal femur defect model. The male SD rats (average weight: 300 g, 8 weeks old) used in the experiment were purchased from the National Tissue Engineering Center in Shanghai, China. The animal experiment process and laboratory animal care were approved by Animal Research Council of the Sixth People's Hospital, Shanghai Jiaotong University School of Medicine. The rats were divided into two groups randomly: B@M and D@M+B@P. Before the operation, the rats were anesthetized by intraperitoneal injection of 2.5% sodium pentobarbital (35 mg/kg body weight), and then a 1 cm linear skin incision was made in the distal femur of each rat, and the muscles were dissected and femoral condyle was exposed. A bicortical defect with a diameter of 3 mm perpendicular to the axis was drilled by a trephine with a diameter of 3 mm (Surgident, Korea). The defect was washed with normal saline to avoid tissue thermal necrosis, and bone fragments were washed out at the same time. Subsequently, the scaffold was implanted in the defect, and the wound was sutured layer by layer. Antibiotic was injected intramuscularly for three consecutive days after surgery to prevent infection. In order to detect the formation of blood vessels at the bone defect, the above-mentioned experimental rats were euthanized at 2, 4, and 8 weeks after the operation, and the blood vessels were perfused with an angiography agent (Microfil, Flowtech, USA). Femoral condyle samples were taken out at 2, 4, and 8 weeks after the operation, fixed in 4% paraformaldehyde solution, and then tested by Micro-CT.

As shown in FIG. 6A, the blood vessel formation at the defect site was studied by microangiography imaging at 2, 4, and 8 weeks after the material was implanted. The contours of the newly formed vascular network and bone defect can be observed in the micro-CT image. The results showed that at 2 weeks after the operation, only a small amount of new blood vessels were formed in the B@M group, while more blood vessels were seen in the D@M+B@P group and extended to the edge of the defect. In the D@M+B@P group, there was the highest blood vessel volume at all time points after surgery, and a large number of blood vessel networks were distributed in the center and around the defect, indicating that the separated distribution of DFO and rhBMP-2 can quickly stimulate angiogenesis and synergistically achieve rapid regeneration of bone tissue.

As shown in FIG. 6B, the results of Micro-CT intuitively show the bone repair process of the defect in the distal femur of rats in each group. In the B@M group only loaded with rhBMP-2, the bone volume in the defect area at 2 weeks was significantly lower than that in the D@M+B@P group, and only a small amount of new bone tissue was formed in the vicinity of the undefected bone depending on the induction ability; and the repair was complete at 8 weeks. Compared with the B@M group, in the D@M+B@P group, a large number of bone trabeculae at 2 weeks were formed after surgery, and bone reconstruction was accomplished quickly at 4 weeks, proving that the separated distribution of DFO and BMP-2 can quickly stimulate bone repair and achieve bone regeneration.

### Example 8

This example relates to the immunofluorescence staining of the osteogenic-related protein Col I for in situ repair of rat femoral defect with composite scaffold

The materials were implanted in the distal femur of the rat, and then the whole femur samples were taken out at 2 weeks, 4 weeks, and 8 weeks for decalcification, paraffin embedding and sectioning. The bone formation was observed by immunofluorescence staining of the osteogenic-related protein Col I to analyze the bone formation effect of each group of scaffolds in vivo. The specific experimental methods are as follows.

Paraffin embedding and sectioning: the sample was soaked in 4% neutral paraformaldehyde solution, placed in a refrigerator at 4° C. for 1 week, and then soaked in 10% EDTA to decalcify the sample until the sample was completely softened. The sample was dehydrated by using gradient ethanol soaking method. The dehydrated sample was placed in a mold, soaked in paraffin at 60° C. for 3 hours, then cooled and embedded to obtain a paraffin block, which was then cut into sections with a thickness of 4.5 μm.

Immunohistochemical staining: The section was soaked in xylene, absolute ethanol, ethanol and pure water for deparaffinization and rehydration and then antigen repair with citric acid buffer was performed, the sample was incubated with 10% goat serum at room temperature for 60 minutes for blocking. Then the sample was incubated overnight at 4° C. with the primary antibody solution prepared according to the antibody instruction. The reaction was quenched by PBST and the sample was washed. The sample was incubated with the HRP-labeled goat anti-mouse IgG secondary antibody at 37° C. for 30 minutes and then the reaction was quenched by PBST and the sample was washed. The sample was incubated in the darkness with DAB chromogenic kit until the tissue appears brown, then rinsed with running water for 10 minutes and stained with hematoxylin. The sample was soaked in 95% ethanol for 1 minute for 2 times, in anhydrous ethanol for 2 minutes for 2 times, and in xylene for 2 minutes for 2 times. Finally, the slide was covered with resin and glass slides. The sample was observed and photographed with an inverted microscope.

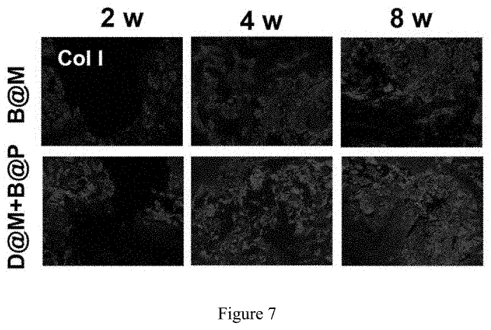

As shown in FIG. 7, the expression of type 1 collagen of the cells over time on the two groups of scaffolds loaded with rhBMP-2 increased significantly. Compared with the B@M group, in the D@M+B@P group, there are more abundant expression of Type 1 collagen in the later stage, indicating that the combination of the hypoxia-simulating drug DFO and bone morphogenetic protein BMP-2 has a better osteogenic effect.

All documents mentioned in the present disclosure are cited as references in this application, as if each document is individually cited as a reference. In addition, it should be understood that after reading the above teaching content of the present disclosure, those skilled in the art can make various changes or modifications to the present disclosure, and these equivalent forms also fall within the scope defined by the appended claims of the present application.

1. A composite scaffold loaded with DFO and rhBMP-2, wherein the composite scaffold contains a matrix, a PEGS gel layer and rhBMP-2, wherein the matrix is an MBG scaffold grafted with DFO on the surface;
the PEGS gel layer is carried on the surface of the matrix; and
rhBMP-2 is contained inside the PEGS gel layer.

2. The composite scaffold of claim 1, wherein the MBG scaffold is a hierarchical pore MBG scaffold with 200 μm-500 μm macropores, 1-3 μm micropores and 2-5 nm mesopores.

3. The composite scaffold of claim 1, wherein the iron ion chelating capacity of the composite scaffold is 5-20 μmol/g.

4. The composite scaffold of claim 1, wherein the thickness of the PEGS gel layer is 1-2 μm.

5. The composite scaffold of claim 1, wherein the loading amount of the rhBMP-2 is 0.005-0.1 μg of rhBMP-2 per mg of scaffold.

6. A preparation method of the composite scaffold of claim 1, wherein the preparation method comprises the following steps:
i) providing a MBG scaffold and PEGS prepolymer including azidated PEGS prepolymer and alkynylated PEGS prepolymer;
ii) grafting DFO on the surface of the MBG scaffold to obtain a matrix;
iii) mixing the azidated PEGS prepolymer with rhBMP-2 to obtain a mixture; coating the mixture on the surface of the matrix obtained in step ii); and then coating the alkynylated PEGS prepolymer to form PEGS gel layer with rhBMP-2 loaded inside, thereby obtaining the composite scaffold;
or coating the azidated PEGS prepolymer solution on the MBG-DFO scaffold and then coating the alkynylated PEGS prepolymer solution to form a PEGS gel isolation layer, and then loading rhBMP-2 to form PEGS gel layer with rhBMP-2 loaded inside, thereby obtaining the composite scaffold.

7. The preparation method of claim 6, wherein in step ii), DFO is grafted onto the surface of the MBG scaffold by the following steps:
ii-1) reacting MBG scaffold with 3-aminopropyltrimethoxysilane (APTMS) to obtain an MBG scaffold with aminated surface, MBG-NH₂;
ii-2) reacting MBG-NH₂ with glutaraldehyde to obtain an intermediate product, MBG-CHO scaffold;
ii-3) reacting MBG-CHO scaffold with DFO, and grafting DFO on the surface of the MBG scaffold to obtain the MBG-DFO scaffold.

8. The preparation method of claim 6, wherein the azidated PEGS prepolymer is obtained by the following steps:
(a-1) reacting PEG with sebacoyl dichloride and triethylamine to obtain sebacoyl dichlorinated PEG;
reacting sebacoyl dichlorinated PEG with glycidol and triethylamine to obtain a long-chain monomer with a ring at both ends;
reacting the monomer with sebacic acid and tetrabutylammonium bromide through ring-opening reaction to obtain PEGS molecules with exposed hydroxyl in the side chain, labeled as HPEGS;
reacting HPEGS with maleic anhydride to obtain maleic acid-functionalized PEGS, labeled as HPEGS-M;
(a-2) adding 3-azidopropylamine and triethylamine to the separation product of dicyclohexylcarbodiimide, N-hydroxysuccinimide and HPEGS-M to obtain the azidated PEGS prepolymer, labeled as HPEGS-Az.

9. The preparation method of claim 8, wherein the alkynylated PEGS prepolymer is obtained by the following steps:
(a-1) reacting PEG with sebacoyl dichloride and triethylamine to obtain sebacoyl dichlorinated PEG;
reacting sebacoyl dichlorinated PEG with glycidol and triethylamine to obtain a long-chain monomer with a ring at both ends;
reacting the monomer with sebacic acid and tetrabutylammonium bromide through ring-opening reaction to obtain PEGS molecules with exposed hydroxyl in the side chain, labeled as HPEGS;
reacting HPEGS with maleic anhydride to obtain maleic acid-functionalized PEGS, labeled as HPEGS-M;
(a-2) reacting HPEGS-M with dicyclohexylcarbodiimide and N-hydroxysuccinimide, and then adding aminated diphenylcyclooctyne and triethylamine to react to obtain the alkynylated PEGS prepolymer, labeled as HPEGS-DBCO.

10. A method for repairing bone tissue comprising the step of administering of the composite scaffold of claim 1 to a subject in need thereof.

11. A composition carrier containing the composite scaffold of claim 1 and a growth factor, or drug.
